(19) United States
(12) Patent Application Publication
(10) Pub. No.: US 2010/0087664 A1
(43) Pub. Date: Apr. 8, 2010

(54) PREPARATION OF CITALOPRAM AND SALTS THEREOF

(76) Inventors: Ravindra Vedantham, Hyderabad (IN); Raveendra Reddy Chinta, Chittoor (IN); Venkata Naga Kali Vara Prasada Raju Vetukuri, Hyderabad (IN); Ambaiah Boini, Medak (IN); Veera Madhavi Sundaraneedi, Hyderabad (IN); Rama Krishna Venkata Medisetti, Visakhapatnam (IN); Kushal Surajmal Manudhane, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/573,930

(22) Filed: Oct. 6, 2009

Related U.S. Application Data
(60) Provisional application No. 61/141,356, filed on Dec. 30, 2008.

(30) Foreign Application Priority Data
Oct. 7, 2008 (IN) 2466/CHE/2008

(51) Int. Cl. C07D 307/00

(52) U.S. Cl. 549/467

(57) ABSTRACT

Process for the preparation of citalopram and its salts, comprising reacting magnesium (5-cyano-2-(4-fluorobenzoyl)phenyl)methanolate bromide with 3-N,N-dimethylaminopropyl magnesium halide in a mixture of solvents, obtaining a diol intermediate in the form of its acid addition salt, and reacting the acid addition salt with sulfuric acid in an aqueous medium.

The drug having the adopted name “citalopram” has a chemical name 1-(3-dimethylaminopropyl)-1-(4-fluorophenyl)-1,3-dihydroisobenzofuran-5-carbonitrile, and is represented structurally by Formula I.

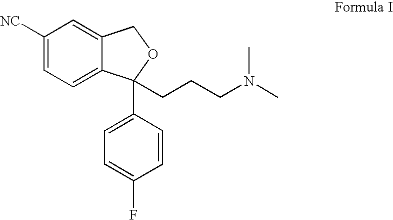

Citalopram is a selective serotonin reuptake inhibitor and is useful in the treatment of depression. Citalopram is commercially available in products containing the drug in the form of its hydrobromide salt.

Citalopram was first disclosed in German Patent 2657013, corresponding to U.S. Pat. No. 4,136,193. This patent outlines process for the preparation of citalopram from the corresponding 5-bromo derivative.

U.S. Pat. No. 4,650,884 discloses a diol intermediate having a chemical name 4-[4-(dimethylamino)-1-(4′-fluorophenyl)-1-hydroxybutyl]-3-(hydroxymethyl)-benzonitrile (Formula II) along with its preparation and use as an intermediate in the preparation of citalopram.

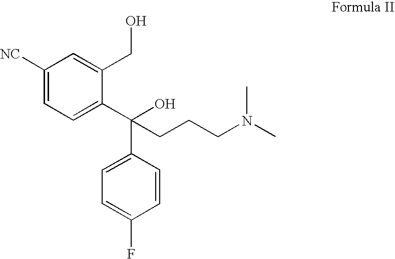

The process described in U.S. Pat. No. 4,650,884 involves reaction of 5-cyanophthalide with 4-fluorophenyl magnesium bromide, followed by reaction with 3-N,N-dimethylaminopropyl magnesium chloride, to obtain the diol intermediate, which is then subjected to a ring closure reaction in the presence of 96% sulphuric acid in a toluene medium to obtain citalopram.

Processes for the preparation of citalopram and its intermediates have also been described in International Application Publication Nos. WO 98/019511, WO 98/019512, and WO 98/019513, and in other documents.

There is an ongoing need for simplified processes for preparing citalopram and its salts, which may be suitable for large scale production in high purity and yield.

### SUMMARY

Aspects of the present invention relate to processes for the preparation of citalopram and its pharmaceutically acceptable salts, embodiments comprising:

(a) reacting 5-cyanophthalide with 4-fluorophenyl magnesium bromide in a mixture of solvents to obtain magnesium (5-cyano-2-(4-fluorobenzoyl)phenyl)methanolate bromide;

(b) reacting magnesium (5-cyano-2-(4-fluorobenzoyl)phenyl)methanolate bromide obtained in (a) with 3-N,N-dimethylaminopropyl magnesium halide in a mixture of solvents, followed by isolation of the diol intermediate of Formula II, in the form of its acid addition salt; and

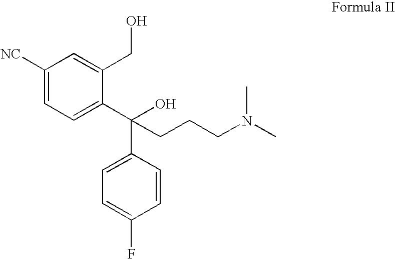

(c) reacting the acid addition salt with sulfuric acid in an aqueous medium, and optionally converting citaolopram to a pharmaceutically acceptable salt.

In embodiments, the product of (a) is reacted in-situ, to avoid isolation of the intermediate magnesium (5-cyano-2-(4-fluorobenzoyl)phenyl)methanolate bromide.

An aspect of the invention provides a process for the preparation of citalopram or its salts, comprising:

a) reacting 5-cyanophthalide with 4-fluorophenyl magnesium bromide in a mixture of solvents to obtain magnesium (5-cyano-2-(4-fluorobenzoyl)phenyl)methanolate bromide;

b) reacting magnesium (5-cyano-2-(4-fluorobenzoyl)phenyl)methanolate bromide with 3-N,N-dimethylaminopropyl magnesium halide in a mixture of solvents, to form the diol intermediate of Formula II;

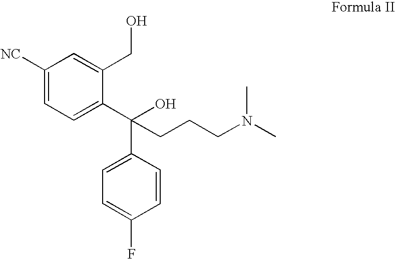

c) reacting the diol intermediate of Formula II with an acid to form an acid addition salt and isolating the acid addition salt; and

d) reacting the acid addition salt with sulfuric acid in an aqueous medium.

An aspect of the invention provides a process for preparing citalopram or its salts comprising:

a) reacting 5-cyanophthalide with 4-fluorophenyl magnesium bromide in a mixture of solvents to obtain magnesium (5-cyano-2-(4-fluorobenzoyl)phenyl)methanolate bromide;

b) reacting magnesium (5-cyano-2-(4-fluorobenzoyl)phenyl)methanolate bromide with 3-N,N-dimethylaminopropyl magnesium halide in a mixture of solvents, to form a diol intermediate of Formula II; and

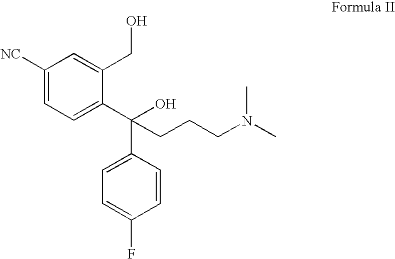

c) reacting the diol intermediate with an acid to form an acid addition salt and isolating the acid addition salt.

An aspect of the invention provides a process for purifying citalopram, comprising treating a solution of citalopram with an adsorbing agent.

### DETAILED DESCRIPTION

Aspects of the present invention relate to processes for preparing citalopram and its pharmaceutically acceptable salts, embodiments comprising:

(a) reacting 5-cyanophthalide with 4-fluorophenyl magnesium bromide in a mixture of solvents, to obtain magnesium (5-cyano-2-(4-fluorobenzoyl)phenyl)methanolate bromide in a mixture of solvents;

(b) reacting magnesium (5-cyano-2-(4-fluorobenzoyl)phenyl)methanolate bromide obtained in (a) with 3-N,N-dimethylaminopropyl magnesium halide in a mixture of solvents, followed by isolation of the diol intermediate of Formula II, in the form of its acid addition salt; and

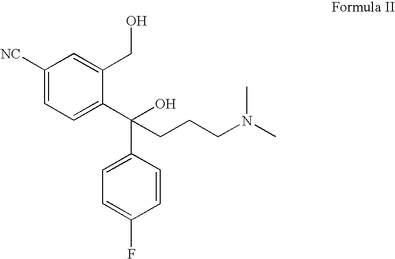

(c) reacting the acid addition salt obtained in (b) with sulfuric acid in an aqueous medium, and optionally converting citalopram to an acid addition salt.

Suitably, the product of (a) is further reacted in situ to avoid isolation of the intermediate magnesium (5-cyano-2-(4-fluorobenzoyl)phenyl)methanolate bromide.

Step (a) involves reaction of 5-cyanophthalide with 4-fluorophenyl magnesium bromide in a mixture of solvents to obtain magnesium (5-cyano-2-(4-fluorobenzoyl)phenyl)methanolate bromide.

4-Fluorophenyl magnesium halide used for the reaction may be prepared in a conventional manner, such as by reacting 4-fluorobromobenzene with magnesium in a suitable solvent, such as ethers including THF (tetrahydrofuran), toluene, benzene, and mixtures thereof. The resultant Grignard reagent may be isolated prior to use in the next reaction.

Suitable temperatures for conducting the reaction range from about 10° C. to about −10° C.

Suitably, the reaction mass containing the intermediate magnesium (5-cyano-2-(4-fluorobenzoyl)phenyl)methanolate bromide can be directly progressed to step (b), without isolation.

Step (b) involves reacting magnesium (5-cyano-2-(4-fluorobenzoyl)phenyl)methanolate bromide obtained in step (a) with 3-N,N-dimethylaminopropyl magnesium halide in a mixture of solvents, followed by isolation of the diol intermediate, in the form of an acid addition salt.

Suitable solvents that can be used in steps (a) and (b) include, but are not limited to: ethers such as diethyl ether, isopropyl ether, tetrahydrofuran, 1,4-dioxane and the like; hydrocarbons such as toluene, xylene, benzene, and the like; halogenated hydrocarbons such as dichloromethane (DCM), ethylene dichloride, and chloroform, aprotic polar solvents such as N,N-dimethylformamide (DMF), dimethylsulfoxide (DMSO), N,N-dimethylacetamide (DMA), and the like or any mixtures thereof.

In embodiments, the reaction steps a), and b) are conducted in a mixture of two or more solvents. In a particular embodiment, the reaction is conducted in a combination of THF, DCM, and toluene.

Suitably, the diol intermediate 4-[4-(dimethylamino)-1-(4′-fluorophenyl)-1-hydroxy-1-butyl]-(3-hydroxymethyl)-benzonitrile, may be isolated in the form of an acid addition salt such as, for example, a hydrochloride, hydrobromide, or hydroiodide salt, after reaction with a suitable acid.

Step c) involves reaction of the acid addition salt of the diol intermediate obtained in step (b) with sulfuric acid in an aqueous medium, and optionally converting it to one of its pharmaceutically acceptable salts.

The cyclization reaction of the diol intermediate may be carried out using an acid, such as, for example, sulphuric acid.

The cyclization may be carried out in an aqueous medium, avoiding the use of organic solvent.

Suitable temperatures for conducting the reaction range from about 70° C. to about 100° C.

After the completion of the reaction, the pH of the reaction mixture may be adjusted to basic by the addition of a suitable base. Suitable bases for adjusting pH include ammonia, sodium or potassium hydroxide, and sodium or potassium carbonate. The product may be extracted into a suitable organic solvent, including: esters, such as, for example, ethyl acetate, n-propyl acetate, n-butyl acetate, and t-butyl acetate; ethers, such as, for example, diethyl ether, dimethyl ether, diisopropyl ether, and methyl t-butyl ether; hydrocarbons, such as, for example, toluene and xylene; halogenated hydrocarbons, such as, for example, chloroform and dichloromethane; and any mixtures thereof.

Suitably, the citalopram obtained may be purified further by stirring a solution of citalopram in an adsorbent that is capable of retaining impurities.

Suitable solvents that can be used for dissolution of citalopram include, but are not limited to: alcohols such as methanol, ethanol, isopropanol, and butanol; esters such as ethyl acetate, n-propyl acetate, n-butyl acetate, and t-butyl acetate; ethers such as diethyl ether, dimethyl ether, diisopropyl ether, methyl t-butyl ether, 1,4-dioxane, and tetrahydrofuran; hydrocarbons, such as toluene and xylene; halogenated hydrocarbons, such as chloroform and dichloromethane; and any mixtures thereof.

Suitable adsorbents include, but are not limited to, activated alumina, silica gel, celite, clay, zeolites, and resins. The absorbent may be separated from the solution by conventional techniques, including filtration.

In embodiments, silica gel may be combined with a solution comprising citalopram in toluene. The resulting mass may be stirred at ambient temperature for a sufficient time to adsorb the impurities. Silica gel is then removed by filtration. The clear filtrate may be evaporated to obtain citalopram in pure form. The purity may range from about 80% to about 99% by weight, as determined using HPLC. This treatment of citalopram with silica gel may optionally be repeated to obtain citalopram having higher purity.

Pure citalopram that is obtained may be converted to any of its pharmaceutically acceptable acid addition salts, by reacting with an acid in a suitable solvent.

Suitable acid addition salts of citalopram include, but are not limited to, hydrohalide salts such as the hydrochloride, hydrobromide, and hydroiodide.

Suitable solvents that can be used include organic solvents, and mixtures thereof. Suitable organic solvents include, but are not limited to: alcohols such as methanol, ethanol, isopropyl alcohol, and n-propanol; ethers such as diethyl ether, dimethyl ether, diisopropyl ether, methyl t-butyl ether, tetrahydrofuran, and 1,4-dioxane; hydrocarbons such as toluene, xylene, n-heptane, cyclohexane, and n-hexane; and any mixtures thereof or their combinations with water in various proportions without limitation.

The treatment with acid may involve stirring of a solution of citalopram in a suitable solvent for a period of about 1 hour to about 10 hours, or longer.

For solid formation to occur, the mass may be maintained further at temperatures lower than the concentration temperatures, such as for example below about 10° C. to about 25° C., for a period of time as required for a more complete isolation of the product.

Optionally, solid formation may be enhanced by methods such as cooling, partial removal of the solvent from the mixture, by adding an anti-solvent to the reaction mixture, or a combination thereof.

Citalopram salts obtained may be further purified by recrystallization from a suitable solvent or mixture of solvents. Suitable solvents include, without limitation thereto: water; alcoholics such as, for example, methanol, ethanol, isopropyl alcohol, n-propanol, and n-butanol; esters such as, for example, ethyl acetate, n-propyl acetate, n-butyl acetate, and t-butyl acetate; ethers such as, for example, diethyl ether, dimethyl ether, diisopropyl ether, methyl t-butyl ether, tetrahydrofuran, and 1,4-dioxane; hydrocarbons, such as, for example, toluene, xylene, n-heptane, cyclohexane, and n-hexane; nitriles such as, for example, acetonitrile and propionitrile; and any mixtures thereof or their combinations with water in various proportions without limitation.

Citalopram and its salt may be dried to lower the content of residual solvents. The drying can be carried out by the methods known in the art such as using a tray dryer, vacuum oven, air oven, fluidized bed dryer, spin flash dryer, flash dryer and the like. Drying may be carried out at temperatures from about 25° C. to about 75° C., with or without vacuum, and in the presence or absence of an inert atmosphere like nitrogen, argon, neon, and helium.

Citalopram salts prepared according to the processes described herein may be in the form of a dry powder having moisture content below about 10% by weight, below about 6% by weight, below about 4% by weight, or substantially free of residual moisture and solvents.

Citalopram and its salts prepared according to the processes described herein may be of high purity, which means at least about 99% by weight pure, and the levels of each impurity may be less than about 1%, about 0.5%, or about 0.1% by weight, as determined using HPLC.

The obtained dry solids may contain small amounts of lump or agglomerated material. A uniform, free flowing solid may be obtained by sieving, air jet milling, pulverization or related methods known in the art. The powder obtained from sieving may be micronized to afford a solid with desired particle size distribution.

Citalopram hydrobromide particles prepared according to certain processes of the present invention have median particle sizes less than about 40 μm, and an average aspect ratio of less than about 2.

The term “particles” refers to individual particles, regardless of whether the particles exist singly or are agglomerated with each other. The term “particle size distribution” refers to the relative amount, e.g., by weight percentage or by number, of each of the different size fractions of particles in a sample. “Median particle size” means the median size of the particles in a sample. Median particle size is expressed in μm and can be measured using various techniques, including a particle size analyzer, such as, for example, using laser light scattering equipment from Malvern Instruments Ltd., Malvern, Worcestershire, United Kingdom. Other types of particle size measuring equipment are also suitable, as will be appreciated by those skilled in the art. The “average aspect ratio” is the mean of average ratio of major axis to minor axis for the particles in a sample.

All percentages and ratios used herein are by weight of the total composition and all measurements made are at about 25° C. and about atmospheric pressure unless otherwise designated. All temperatures are in degrees Celsius unless specified otherwise. As used herein, “comprising” (open ended) means the elements recited, or their equivalent in structure or function, plus any other element or elements which are not recited. The terms “having” and “including” are also to be construed as open ended. As used herein, “consisting essentially of” means that the invention may include ingredients in addition to those recited in the claim, but only if the additional ingredients do not materially alter the basic and novel characteristics of the claimed invention. All ranges recited herein include the endpoints, including those that recite a range “between” two values. Whether so indicated or not, all values recited herein are approximate as defined by the circumstances, including the degree of expected experimental error, technique error, and instrument error for a given technique used to measure a value.

Citalopram or its pharmaceutically acceptable salts obtained by a process of the present invention can be used to prepare its pharmaceutical compositions along with one or more pharmaceutically acceptable excipients.

Pharmaceutical composition comprising citalopram or its pharmaceutically acceptable salts together with one or more pharmaceutically acceptable carriers may be formulated as: solid oral dosage forms, such as, but not limited to: powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the forms of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The compositions may be prepared using techniques such as direct blending, dry granulation, and wet granulation, or by extrusion and spheronization. Compositions may be presented as uncoated, film coated, sugar coated, powder coated, enteric coated or modified release coated.

Pharmaceutically acceptable excipients that find use in the present invention include, but are not limited to, any one or more of: diluents such as starches, pregelatinized starches, lactose, powdered celluloses, microcrystalline celluloses, dicalcium phosphate, tricalcium phosphate, mannitol, sorbitol, sugar and the like; binders such as acacia, guar gum, tragacanth, gelatin, polyvinylpyrrolidones, hydroxypropyl celluloses, hydroxypropyl methylcelluloses, pregelatinized starches, and the like; disintegrants such as starches, sodium starch glycolate, pregelatinized starches, crospovidones, croscarmellose sodium, colloidal silicon dioxide, and the like; lubricants such as stearic acid, magnesium stearate, zinc stearate and the like; glidants such as colloidal silicon dioxide and the like; solubility or wetting enhancers such as anionic or cationic or neutral surfactants; complex forming agents such as various grades of cyclodextrins and resins; release rate controlling agents such as hydroxypropyl celluloses, hydroxymethyl celluloses, hydroxypropyl methylcelluloses, ethyl celluloses, methyl celluloses, various grades of methyl methacrylates, waxes and the like. Other pharmaceutically acceptable excipients that are of use include, but are not limited to, film formers, plasticizers, colorants, flavoring agents, sweeteners, viscosity enhancers, preservatives, antioxidants and the like.

In the compositions of the present invention, citalopram or its pharmaceutically acceptable salt is a useful active ingredient in the range of 10 mg to 100 mg, or 20 mg to 60 mg.

The following examples will further describe certain specific aspects and embodiments, and are provided solely for purposes of illustration. However, one skilled in the art will readily appreciate that the specific methods and results discussed are merely illustrative of the invention and no limitation of the invention is implied.

### Example 1

### Preparation of 4-[4-(dimethylamino)-1-(4′-fluorophenyl)-1-hydroxybutyl]-3-(hydroxymethyl)-benzonitrile hydrobromide

A solution of 4-fluorophenyl magnesium bromide, prepared from 4-fluoro-1-bromobenzene (71.6 g) and magnesium (11.3 g) in THF (100 mL), is added to a suspension of 5-cyanophthalide (50 g) in dichloromethane (150 mL) at 5 to −5° C. and stirred at that temperature for 30 minutes. After the completion of the reaction, a solution of 3-N,N-dimethylaminopropyl magnesium chloride, prepared by reacting 3-N,N-dimethylaminopropyl chloride (65%, 173.6 g) in toluene (200 mL) with magnesium (22.6 g) in THF (75 mL), is added at 5 to −5° C. After completion of the reaction, the reaction is quenched with aqueous ammonium chloride (5%, 250 mL). The mass is filtered and the solid is washed with toluene (50 mL). Water (250 mL) is added to the filtrate. The filtrate is treated with aqueous HBr (47%, 38 mL) at 25-35° C. and stirred for 2 hours. The solid is collected by filtration, washed with water (2×50 mL) and toluene (2×50 mL), and dried at 60-70° C. for 6 hours. (Yield: 110 g).

### Example 2

### Preparation of Citalopram Hydrobromide

To a solution of 4-[4-(dimethylamino)-1-(4′-fluorophenyl)-1-hydroxybutyl]-3-(hydroxymethyl)-benzonitrile hydrobromide (100 g) in water, (1000 mL), concentrated sulphuric acid (46.3 g) is added. The mixture is heated to 100° C. and stirred for 8 hours. The mixture is cooled to 25-35° C. and toluene (500 mL) is added. The pH of the mixture is adjusted to 8-8.5 with aqueous sodium hydroxide (20%, 230 mL) at 25-35° C. The layers are separated. The aqueous layer is extracted with toluene (300 mL). The organic layers are combined and toluene (300 mL) is added. To the combined organic layer, silica gel (100-200 mesh, 50 g) is added and the mass is stirred at 25-35° C. for 30 minutes. The silica gel is removed by filtration and washed with toluene (200 mL). The filtrate is treated with charcoal (10 g) and stirred at 25-35° C. for 30 minutes. The charcoal is removed by filtration and washed with toluene (100 mL). The solvent is distilled under reduced pressure to obtain a residue. The residue is dissolved in isopropyl alcohol (500 mL) and aqueous HBr (47%, 40 g) is added at ambient temperature. The mixture is cooled to 0-5° C. and stirred for 90 minutes. The mass is filtered to collect the precipitated solid. The solid is washed with isopropyl alcohol (75 mL) and dried at 60-70° C. for 6 hours. (Yield: 80 M.

### Example 3

### Purification of Citalopram Hydrobromide

Citalopram hydrobromide (50 g) is combined with a mixture of isopropanol (300 mL) and water (5 mL). The mixture is heated to reflux (about 85° C.) and stirred for 30 minutes. The mixture is filtered and the filtrate is slowly cooled to 0-5° C. The mass is stirred at 0-5° C. for 2 hours. The formed solid is collected by filtration and dried at 60-70° C. for 6 hours under vacuum. (Yield: 47 g).

We claim:

1. A process for the preparation of citalopram or its salts, comprising:
a) reacting 5-cyanophthalide with 4-fluorophenyl magnesium bromide in a mixture of solvents to obtain magnesium (5-cyano-2-(4-fluorobenzoyl)phenyl)methanolate bromide;
b) reacting magnesium (5-cyano-2-(4-fluorobenzoyl)phenyl)methanolate bromide with 3-N,N-dimethylaminopropyl magnesium halide in a mixture of solvents, to form the diol intermediate of Formula II; 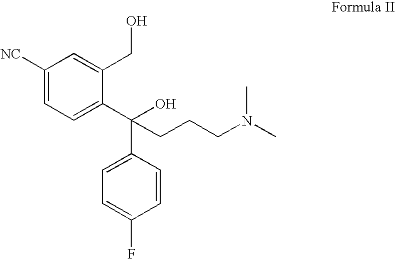
c) reacting the diol intermediate of Formula II with an acid to form an acid addition salt and isolating the acid addition salt; and
d) reacting the acid addition salt with sulfuric acid in an aqueous medium.

2. The process of claim 1, wherein an acid addition salt is a hydrobromide salt.

3. The process of claim 1, wherein a solvent in a mixture comprises an ether, a hydrocarbon, or a halogenated hydrocarbon.

4. The process of claim 1, wherein a mixture of solvents comprises tetrahydrofuran, toluene, and dichloromethane.

5. A process for preparing citalopram or its salts comprising:
a) reacting 5-cyanophthalide with 4-fluorophenyl magnesium bromide in a mixture of solvents to obtain magnesium (5-cyano-2-(4-fluorobenzoyl)phenyl)methanolate bromide;
b) reacting magnesium (5-cyano-2-(4-fluorobenzoyl)phenyl)methanolate bromide with 3-N,N-dimethylaminopropyl magnesium halide in a mixture of solvents, to form a diol intermediate of Formula II; and 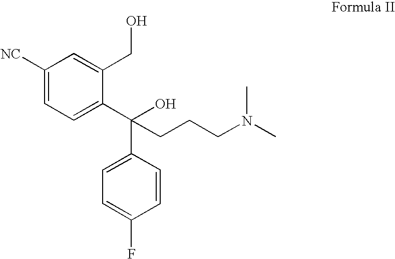
c) reacting the diol intermediate with an acid to form an acid addition salt and isolating the acid addition salt.

6. The process of claim 5, wherein magnesium (5-cyano-2-(4-fluorobenzoyl)phenyl)methanolate bromide is not isolated.

7. The process of claim 5, wherein a solvent in a mixture comprises an ether, a hydrocarbon, or a halogenated hydrocarbon.

8. The process of claim 5, wherein a mixture of solvents comprises tetrahydrofuran, toluene, and dichloromethane.

9. The process of claim 5, wherein the diol intermediate or its salt is isolated from a mixture of solvents.

10. The process of claim 9, wherein the diol intermediate or its salt is isolated from a mixture of dichloromethane, toluene, tetrahydrofuran, and water.

11. A process for the preparation of citalopram, comprising reacting the diol intermediate of Formula II with sulfuric acid in an aqueous medium.

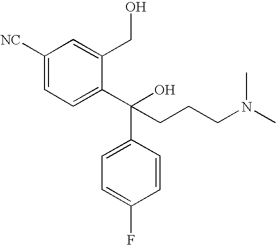

12. The process of claim 11, wherein a molar ratio of sulfuric acid to diol intermediate is about 0.8 to about 1.5.

13. The process of claim 11, wherein citalopram is further converted to a salt.

14. The process of claim 11, wherein citalopram is further converted to a hydrochloride, hydrobromide, or hydroiodide salt.

15. The process of claim 11, wherein citalopram is further converted to a hydrobromide salt.

16. A process for purifying citalopram, comprising treating a solution of citalopram with an adsorbing agent.

17. The process of claim 16, wherein an adsorbing agent comprises activated alumina, silica gel, celite, a clay, a zeolite, or a resin.

18. The process of claim 16, wherein an adsorbing agent comprises silica gel.

19. The process of claim 16, wherein a solution of citalopram includes a hydrocarbon solvent.

20. The process of claim 16, wherein a solution of citalopram comprises toluene.
